(19) United States
(12) Patent Application Publication
(10) Pub. No.: US 2015/0238141 A1
(43) Pub. Date: Aug. 27, 2015

(54) WATCH WITH SEPARATE PROCESSOR AND DISPLAY HOUSING

(71) Applicant: Hing Yin Lai, San Jose, CA (US)

(72) Inventor: Hing Yin Lai, San Jose, CA (US)

(21) Appl. No.: 14/519,112

(22) Filed: Oct. 20, 2014

Related U.S. Application Data
(63) Continuation-in-part of application No. 14/191,411, filed on Feb. 26, 2014, now abandoned.

(51) Int. Cl. A61B 5/00; A61B 5/0205; G04G 17/00; A61B 5/22

(52) U.S. Cl. CPC A61B 5/681; A61B 5/224; A61B 5/02055; G04G 17/00; A61B 5/01

(57) ABSTRACT

A watch includes a watch housing with a display and a battery, wherein the housing has no processor therein. The watch also includes a removable processor module. A wrist band is secured to one side of the watch housing with a recessed slot to receive the removable processor module. The band also has a connector to connect the processor module to the display and the battery.

This application is a continuation in part application Ser. No. 14/191,411 filed Feb. 26, 2014, the content of which is incorporated by reference.

The present invention relates to a watch.

Smart watches are making their move on the marketplace. FIG. 1 shows the Samsung Galaxy Gear, which lets the user take calls, send texts, control the phone's media playback and do a few other things without taking the handset out of the user pocket. According to a review at http://www.wired.co.uk/reviews/gadgets/2013-10/samsung-galaxy-gear-review, the Gear is chunky and heavy. The display has a resolution of 320×320 pixels for viewing info and displaying the pictures that the user can take via the 1.9-megapixel camera in the strap. Incoming calls and texts are flashed up on the Gear's screen as they come into the phone, together with contact details and a contact photograph if the user has them. Taking the call feels a bit weird though, and the user may find the herself shouting at the her wrist as the user tries to compensate for the speaker, which doesn't cope too well with background noise. Battery life is a long way short of what the user'd expect from a standard watch—instead of months the user get a day or two (and that's if the user switch it off when the user're not using it).

### SUMMARY

In one aspect, a watch includes a watch housing with a display and a battery, wherein the housing has no processor therein. The watch also includes a removable processor module. A wrist band is secured to one side of the watch housing with a recessed slot to receive the removable processor module. The band also has a connector to connect the processor module to the display and the battery.

In another aspect, a method is disclosed for operating a watch having a housing with a display and a battery without a processor therein, the watch having a band coupled to one side of the watch housing. The method includes inserting a removable processor module into a recessed slot; connecting the processor module to the display and the battery; and communicating with a remote device from the processor module.

Implementations of the above aspects can include one or more of the following. A two pin charger port can be used to recharge the battery. The recessed slot can be inside the band, on top of the band, or on bottom of the band. Sensors can be placed on the band for detecting muscle strength, CO2, temperature, and pulse rate. A remote receiver can be placed on a remote device which can be a movable toy such as a toy car or helicopter.

Advantages of the system may include one or more of the following. By moving the processor and sensor electronics outside of the watch housing, more space is created to put a high capacity battery in the housing or to put a high resolution display in the housing. Alternatively, the watch housing can be made low profile to be more stylish given the same battery capacity. The watch provides enhanced convenience when users check for notifications, including emails, texts, updates, and so on. The watch delivers notifications straight to the user's wrist, and allows for the wearer to not have to check their phone so often. The watches may help people with their health and fitness tracking. The removable module may be able to replace smartphones with the use of a SIM card, and can make or take calls on their own without having to be paired with a smartphone.

### BRIEF DESCRIPTION OF THE DRAWINGS

FIG. 1 shows a conventional watch.

FIG. 2 shows an exemplary embodiment for side insertion of a removable smart controller in a wrist band.

FIG. 3 shows an exemplary embodiment for top insertion of a removable smart controller into the wrist band.

FIG. 4 shows an exemplary control of a robot with a hand.

FIG. 5 shows a cloud-based hand-control of the robot of FIG. 5.

FIG. 6 shows a block diagram of a removable processor system.

### DESCRIPTION

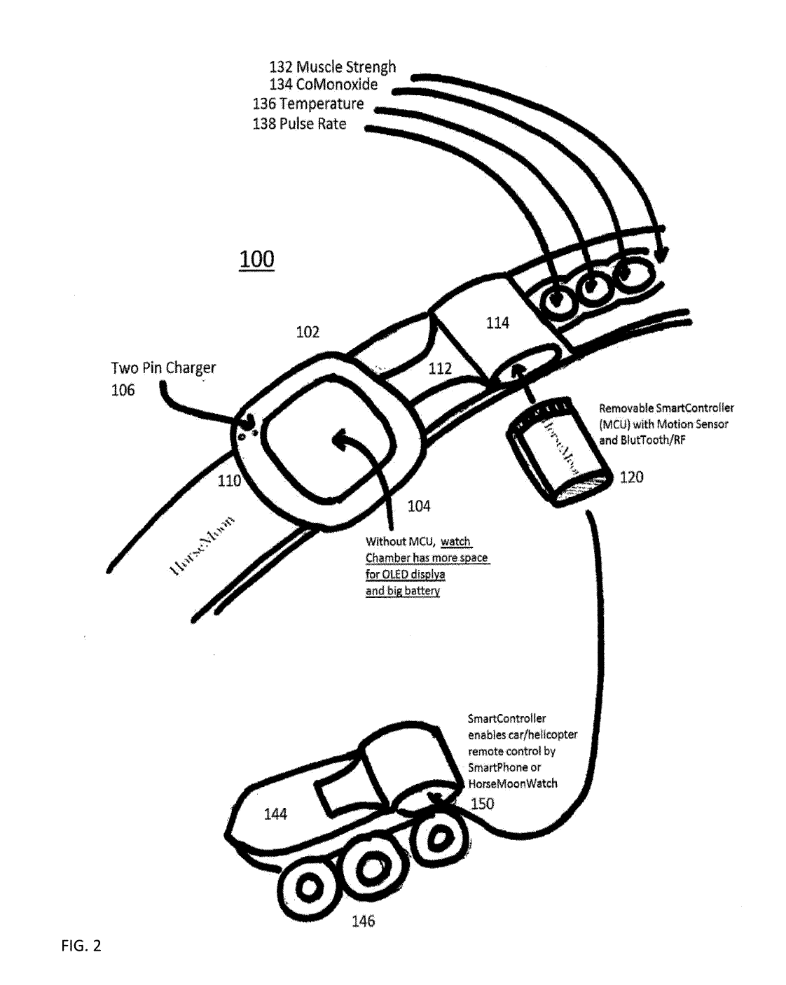

FIG. 2 shows an exemplary embodiment for side insertion of a removable smart controller in a wrist band. A watch 100 includes a watch housing or chamber 102 and a display 104, but no processor therein. Without processor and support electronics, the chamber 102 can be low profile. Alternatively, the chamber 102 can be the same size as conventional casing but now has more room for high capacity battery to provide long operating time for the watch. The battery is recharged using a two pin charger port 106. The chamber 102 is connected to halves 110 of a wrist band. A recessed slot, opening or chamber 114 inside the band 110 provides space to receive a removable smart controller 120 with motion sensor and Bluetooth radio in one embodiment. The controller 120 is connected to the display 104 in the chamber 102 via a connector or cable 112. The controller 120 is also connected to various sensors mounted on the wrist band, including muscle strength sensor 132, carbon monoxide sensor 134, temperature sensor 136, and pulse rate 138.

In one embodiment, a horizontal bar-like muscle strength sensor detects applied force. This sensor is capable of sliding over a couple of vertical posts each containing a position adjustment mechanism between the post and that muscle strength sensor. The middle portion of the muscle testing mechanism contains a number of tensile sensors such as a piezo-electric sensor. When the user applies force through a clearly marked grip area of the mechanism, the signal from the tensile sensors is transmitted through a computational block to a display unit. Additionally, pull force testing units are provided on one or preferably both vertical posts that are designed to allow adjustment of their position as well along the posts. Each unit contains a spring-loaded housing and a force displaying gauge activated by pulling on a flexible link such as a cable or a rope. Depressing of a spring via a lever releases the housing and allows repositioning along the post.

In another embodiment, a carbon monoxide sensor is used. The carbon monoxide sensor can include a zirconia substrate, a pair of platinum electrodes, and another pair of gold electrodes. The sensor determines a carbon monoxide concentration based on a difference between a potential difference of the platinum electrodes and a potential difference of the gold electrodes. However, the sensor is influenced by a partial pressure of oxygen in a gas. Therefore, even though a carbon monoxide concentration is constant, the sensor may give a varying value of the carbon monoxide concentration depending on the partial pressure of oxygen.

In another embodiment, a temperature sensor is used. A number of temperature sensors, based on a variety of different physical measurement principles, can be used for conducting a temperature measurement. Especially popular are electric temperature sensors, for example PTC sensors (positive temperature coefficient sensor) or NTC sensors (negative temperature coefficient sensor), or thermocouples, which have a very simple design and are inexpensive to produce. The actual sensor element can be an electric ohmic resistor, which changes with the temperature. Thermocouples consist of a contact point of two different metals, and this contact point generates a thermal stress when the temperature changes. In the simplest case sensors of this type are produced as a sensor pill with at least two connecting wires. The evaluation of the electric signal is usually performed in an electronic unit, in which the sensor element is a part of a bridge circuit.

In one embodiment, the controller 120 communicates with a receiver module 150 on a remote device 144 which can be a remote car with wheels 146 or a remotely controlled helicopter, for example. The user can transmit commands to cause the remote device to move to a predetermined location or to actuate the remote device to take a picture or a video, among others.

In another embodiment, a heart rate sensor monitors the EKG (electrocardiogram) wave produced by the heartbeat. The EKG is picked up by a pair of electrodes, usually located on the chest, is placed on one side facing the wrist and one finger. As is well known, this EKG wave is of a complex nature and includes as a component thereof what is known as the R wave or pulse. In the present invention, this R wave is selectively amplified to a predetermined level, the amplifier R wave then being used to trigger a monostable multivibrator that produces a square wave of a known and fixed duration for each beat of the heart. The duration of the square wave is set equal to the R to R intervals of the prescribed or specified heart rate and what is then detected is the interval from the end of one square wave to the onset of the next. This is done by feeding the square wave through a one-way diode to a parallel resistor-capacitor circuit with a short time constant. The capacitor almost immediately charges to nearly the full value of the square wave and holds this value until the end of the wave. It then starts to discharge through the resistor. But if the next square wave comes along within a short period of time (5 to 10 milliseconds), very little of its charge will have dissipated so that a basically DC voltage equal to the peak amplitude of the multivibrator square wave is developed. This DC voltage is fed through a high resistance to one side of the diode, the other side being connected to the output of an audio oscillator whose base to peak amplitude just equals this DC voltage. If an earphone is placed on the DC side of the diode, no sound will be heard because even at the peak of the audio signal the amplitude just equals the DC back bias and thus no current can flow. If, now, the heart rate decreases, thereby increasing the time between the end of one square wave and the onset of the next, the above-said storage capacitor discharges further, thereby lowering the DC back bias to momentarily allow some audio signal through the diode.

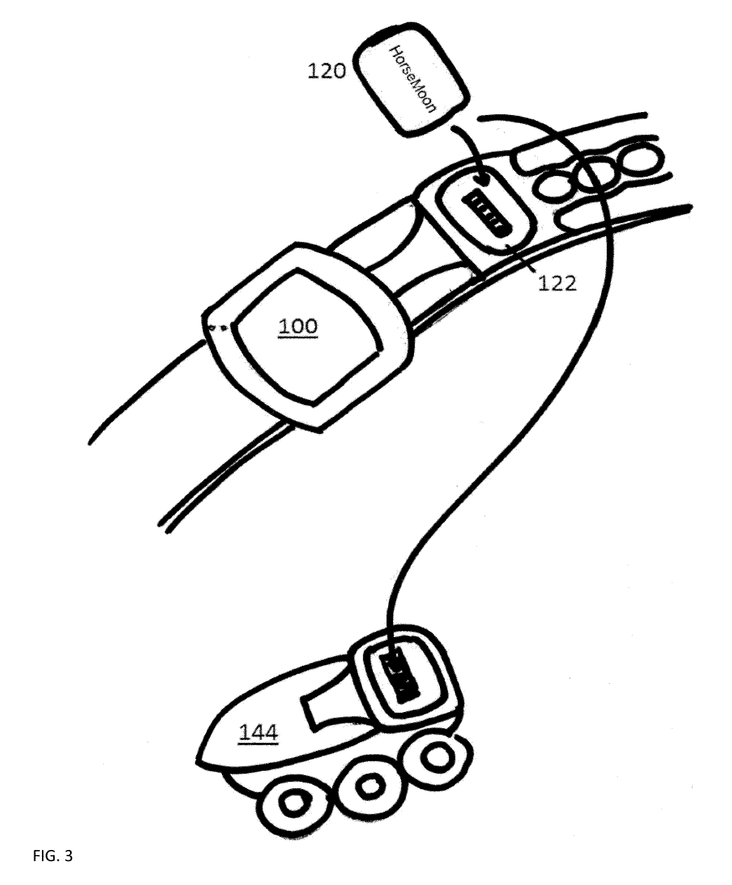

FIG. 3 shows an exemplary embodiment for top insertion of a removable smart controller into the wrist band. In this embodiment, a pocket 122 on top or bottom of the band 110 provides space to receive the removable smart controller 120 with motion sensor and Bluetooth radio in one embodiment. The controller 120 is connected to the display 104 in the chamber 102 via a connector or cable 112. The controller 120 is also connected to various sensors mounted on the wrist band, including muscle strength sensor 132, carbon monoxide sensor 134, and temperature sensor 136, and pulse rate 138.

The system may be implemented in hardware, firmware or software, or a combination of the three. Preferably the invention is implemented in a computer program executed on a programmable computer having a processor, a data storage system, volatile and non-volatile memory and/or storage elements, at least one input device and at least one output device.

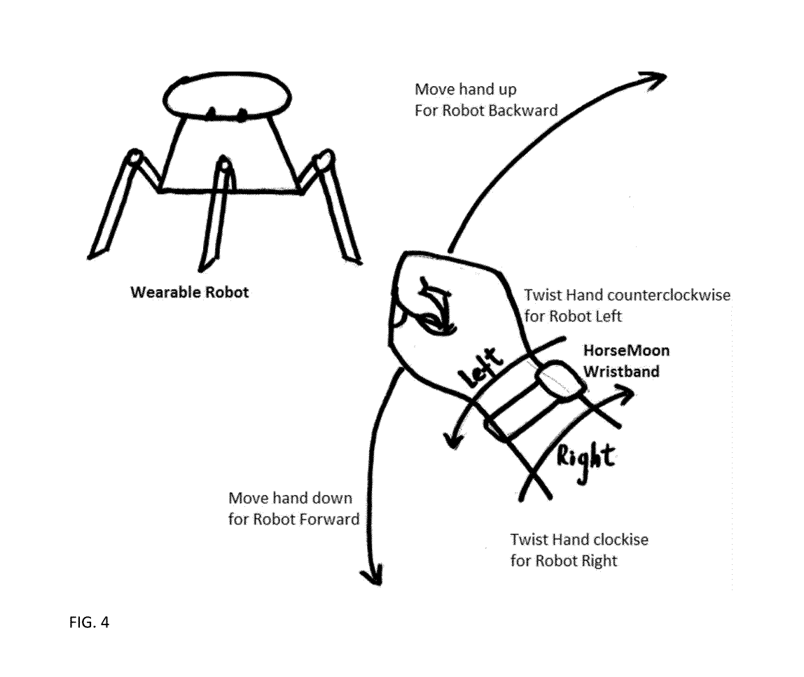

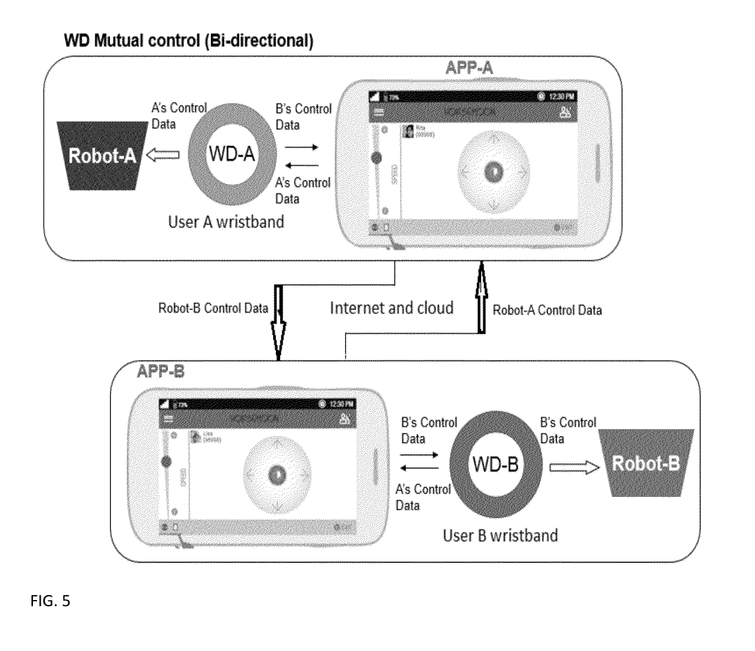

FIG. 4 shows an exemplary control of a robot with a hand. One embodiment uses the wearable device embedded in wristband to detect hand movement for robotic control, while FIG. 5 shows a cloud-based hand-control of the robot of FIG. 4. In one embodiment, the removable smartcontroller has a MCU (micro controller unit) on wristband. It has motion sensor to detect hand movement. The user can:
Move hand up to control robotic forward direction
Move hand down to control robotic backward direction
Twist hand counterclockwise to control robotic left direction
Twist hand clockwise to control robotic right direction.

In one embodiment, a Health Social Network App enables Bi-directional wearable robotic control sharing through the internet cloud. Turning now to the example of FIG. 5, the Health Social Network App in smartphone allow bi-directional wearable robot control through internet.

In one embodiment, when a User A wants to control a Remote user B's robot, User A will request for control and B accepts the control. Once user B accepts the control, User A can start to use his App or wristband to remote control user B robot around the global in different geographic location in real time. Meanwhile, User B can also start to use his App or wristband to remote control user A's robot in the same time. This scheme achieves mutual and bi direction robot control sharing globally through internet cloud. The cloud stores commands from App A and App B and determines the appropriate robot A or B to relay the commands thereto. In this manner, user A and B can control remote robots after appropriate authentication.

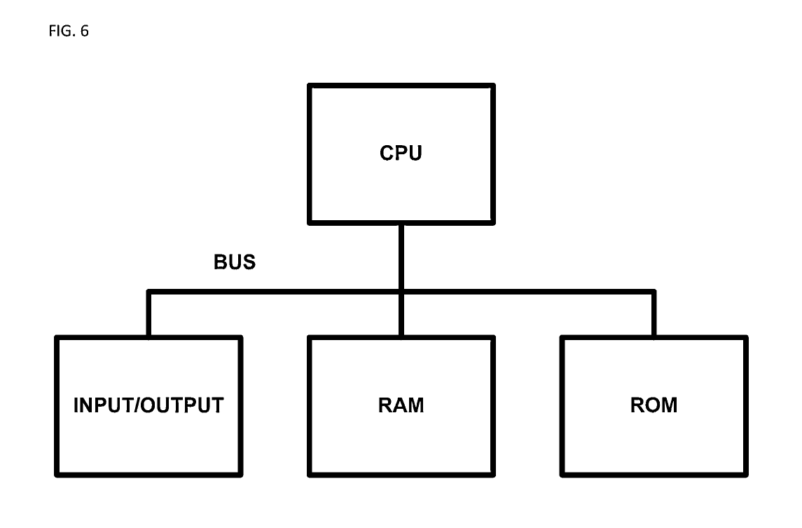

By way of example, FIG. 6 shows a block diagram of a removable processor module to support the system. The computer preferably includes a processor, random access memory (RAM), a program memory (preferably a writable read-only memory (ROM) such as a flash ROM) and an input/output (I/O) controller coupled by a CPU bus. The computer may optionally include a hard drive controller which is coupled to a hard disk and CPU bus. Hard disk may be used for storing application programs, such as the present invention, and data. Alternatively, application programs may be stored in RAM or ROM. I/O controller is coupled by means of an I/O bus to an I/O interface. I/O interface receives and transmits data in analog or digital form over communication links such as a serial link, local area network, wireless link, and parallel link. Optionally, a display, a keyboard and a pointing device (mouse) may also be connected to I/O bus. Alternatively, separate connections (separate buses) may be used for I/O interface, display, keyboard and pointing device. Programmable processing system may be preprogrammed or it may be programmed (and reprogrammed) by downloading a program from another source (e.g., a floppy disk, CD-ROM, or another computer).

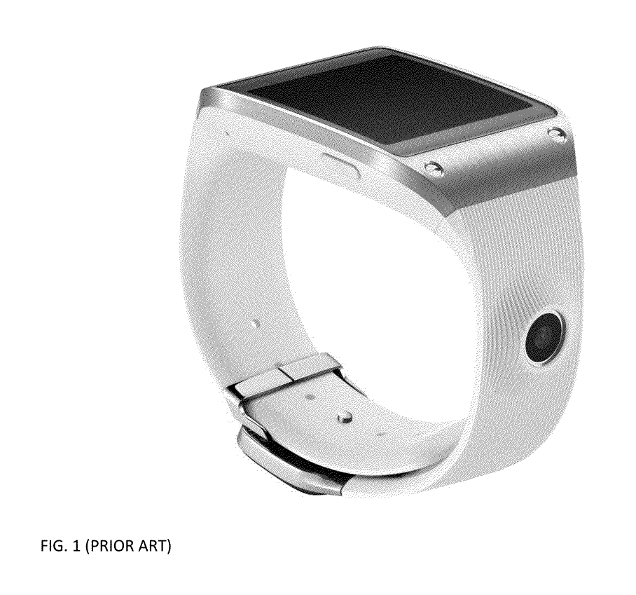

In one embodiment, the device can be a phone such as the iPhone. The iPhone has a 3G cellular transceiver devices, ROM and RAM. For display, the iPhone has a 3.5 inches (8.9 cm) liquid crystal display (320×480 pixels) HVGA, acting as a touch screen that has been created for the use with one finger or multiple fingers. No stylus is needed nor can it be used, since the touch screen is not compatible with it. For the text input, the data entry system shown in FIGS. 1-3 can be used. The data entry system can work with the iPhone's built-in spell-checker, predictive word capabilities and a dynamic dictionary that retains new words. The predictive words capabilities have been integrated with the data entry system described above so that the user does not have to be perfectly accurate when typing—unwitting swipe on the edges of the nearby letters on the keyboard will be corrected when possible.

In another embodiment, the device can be a music player such as the iPod. All iPods (except the current iPod Shuffle and iPod Touch) have five buttons and the later generations have the buttons integrated into the click wheel—an innovation that gives an uncluttered, minimalist interface. The buttons perform basic functions such as menu, play, pause, next track, and previous track. Other operations, such as scrolling through menu items and controlling the volume, are performed by using the click wheel in a rotational manner. The current iPod Shuffle does not have any controls on the actual player; instead it has a small control on the earphone cable, with volume-up and -down buttons and a single button for play/pause, next track, etc. The iPod Touch has no click-wheel; instead it uses a 3.5″ touch screen in addition to a home button, sleep/wake button and (on the second and third generations of the iPod touch) volume-up and -down buttons. The user interface for the iPod touch is almost identical to that of the iPhone. Differences include a slightly different Icon theme and lack of the Phone application on the iPod touch. Both devices use the iPhone OS.

In yet another embodiment, the device can be a tablet computer such as the iPad. The footprint of the iPad is roughly the same as that of a netbook though the iPad is wider because its display uses the “conventional” 4:3 aspect ratio. However, since the iPad is a tablet and not a clamshell, it is thinner than any netbook, and lighter, too. While most netbooks are in the 2.5 pound range, the iPad weighs 1.5 pounds and is a scaled-up version of the iPhone. As a result, the iPad does not need very powerful (and power-hungry) hardware to do what it does quickly and effortlessly.

Each computer program is tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

The invention has been described herein in considerable detail in order to comply with the patent Statutes and to provide those skilled in the art with the information needed to apply the novel principles and to construct and use such specialized components as are required. However, it is to be understood that the invention can be carried out by specifically different equipment and devices, and that various modifications, both as to the equipment details and operating procedures, can be accomplished without departing from the scope of the invention itself.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. A watch, comprising:
a watch housing, including:
a display; and
a battery, wherein the housing has no processor therein;
a removable processor module;
a band coupled to one side of the watch housing with a recessed slot to receive the removable processor module and to connect the processor module to the display and the battery.

2. The watch of claim 1, comprising a two pin charger port to recharge the battery.

3. The watch of claim 1, wherein the recessed slot is inside the band, on top of the band, or on bottom of the band.

4. The watch of claim 1, comprising a muscle strength sensor mounted on the band.

5. The watch of claim 1, comprising a carbon monoxide sensor mounted on the band.

6. The watch of claim 1, comprising a temperature sensor mounted on the band.

7. The watch of claim 1, comprising a pulse rate sensor mounted on the band.

8. The watch of claim 1, comprising sensors for detecting muscle strength, CO2, temperature, and pulse rate on the band.

9. The watch of claim 1, comprising a remote receiver on a remote device.

10. The watch of claim 9, wherein the remote device comprises a movable toy.

11. A method for operating a watch having a housing with a display and a battery without a processor therein, the watch having a band coupled to one side of the watch housing, the method comprising:
inserting a removable processor module into a recessed slot;
connecting the processor module to the display and the battery; and
communicating with a remote device from the processor module.

12. The method of claim 11, comprising charging the battery with a two pin charger port.

13. The method of claim 11, wherein the recessed slot is inside the band, on top of the band, or on bottom of the band.

14. The method of claim 11, comprising sensing muscle strength mounted on the band.

15. The method of claim 11, comprising sensing a carbon monoxide mounted on the band.

16. The method of claim 11, comprising sensing temperature mounted on the band.

17. The method of claim 11, comprising sensing pulse rate mounted on the band.

18. The method of claim 11, comprising detecting muscle strength, CO2, temperature, and pulse rate from the band.

19. The method of claim 11, comprising communicating with a remote receiver on a remote device.

20. The method of claim 19, wherein the remote device comprises first and second movable robots, each controlled by a remote smart phone communicating over a cloud.
